(19) United States
(12) Patent Application Publication
(10) Pub. No.: US 2004/0204692 A1
(43) Pub. Date: Oct. 14, 2004

(54) Implantable vascular access device

(76) Inventor: Kenneth Eliasen, East Bridgewater, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 10/412,515

(22) Filed: Apr. 11, 2003

(51) Int. Cl. A61M 31/00

(52) U.S. Cl. 604/288.02

(57) ABSTRACT

A configurable implantable access port is provided having as individual interchangeable components a chamber body defining a fluid chamber, a body component, and a septum. The individual components may be assembled by mechanical feature, adhesive, or welding to provide a complete access port. Preferably at least the body component is provided having more than one size or geometric configuration. The access port may be tailored before implantation to a specific application by combining the chamber body and septum with a body component suitable to the specific application.

### FIELD OF INVENTION

The present invention relates generally to a subcutaneously implantable vascular access port. More specifically, the present invention relates to an implantable access port having a needle-penetrable, self-sealing septum which affords repeated access to a fluid cavity in communication with a catheter. The implantable access port is an assembly of interchangeable components rendering the implantable access port susceptible to multiple configurations.

### BACKGROUND OF THE INVENTION

Access portals, or ports, provide a convenient method to repeatedly deliver medicants to remote areas of the body without utilizing surgical procedures. The port is totally implantable within the body, and permits the infusion of medications, parenteral solutions, blood products, and other fluids. The port may also be used for blood sampling.

Known ports typically include a chamber accessible through a self-sealing septum. Septums of the prior art vary in shape, from a wafer-like cylindrical block of silicone to a pre-molded septum of U.S. Pat. No. 4,802,885 to Weeks et al. The pre-molded septum of U.S. Pat. No. 4,802,885 includes opposed convex surfaces and a peripheral ledge.

In common practice, a caregiver locates the septum of the port by palpitation. Port access is accomplished by percutaneously inserting a needle, typically a non-coring needle, perpendicularly through the septum of the port and into the chamber. The drug or fluid is then administered by bolus injection or continuous infusion. Ordinarily the fluid flows through the chamber, into a catheter and finally to the site where the fluid is desired. Except for the septum, traditional ports are constructed from all-metal or all-plastic. Each type of construction has unique advantages and disadvantages.

All-metal constructions have the advantages that they maintain a septum in a self-sealing fashion after repeated percutaneous injections. Additionally, all-metal constructions, such as titanium, or stainless steel provide a port which is both biocompatible and compatible with the injected fluid.

However, all-metal constructions present the disadvantages that they are relatively heavy, difficult to fabricate and relatively expensive. Additionally, all-metal ports produce large Magnetic Resonance Imaging (MRI) artifacts. On the other hand, all-plastic ports have the advantages that they are inexpensive to construct, light in weight, and do not create an MRI artifact. However, ports constructed from plastic have the disadvantage that infused fluids may react with the plastic body of the port. All-plastic ports contain the disadvantage that they cannot maintain a sealing engagement with the septum after repeated percutaneous injections. Additionally, all-plastic ports are susceptible to nicks and scratches on the interior surface by the accessing needle. These nicks and scratches could lead to nidus, blood clots, or precipitation formations.

Efforts have been made to combine the advantages of all-metal ports with all-plastic ports. For example, in U.S. Pat. No. 4,802,885 to Weeks et al., a metal reservoir having a chamber sealed by a pre-formed silicone septum is jacketed by a single piece of a silicone elastomer. However, all-metal ports jacketed by a single piece of elastomer have significant shortcomings. These shortcomings include quality control problems during manufacturing, and expensive molding processes.

Other efforts have focused on providing a multiple piece all-plastic housing in cooperation with an open metal cup to sealingly engage a septum. For example, see U.S. Pat. No. 5,213,574 to Tucker. This design has shortcomings associated with it, including defects in the plastic housing which may cause an improperly sealed septum. Once the septum is improperly sealed the entire port must be discarded.

Therefore a need has arisen for an access port device which addresses the problems of prior port devices.

A variety of implantable devices, known as subcutaneous access ports, are utilized to deliver fluids to or to withdraw fluids from the bloodstream of a patient. Such access ports typically include a needle-impenetrable housing which encloses one or more fluid cavities and defines for each such fluid cavity an access aperture communicating through the housing on the side thereof which is adjacent to the skin of the patient when the access port is implanted in the body. A needle-penetrable septum is received in and seals each access aperture. Exit passageways located in an outlet stem communicate with each of the fluid cavities for dispensing medication therefrom to a predetermined location in the body of the patient through an implanted catheter attached to the access port.

Once the access port and the catheter have been implanted beneath the skin of a patient, quantities of medication or blood may be dispensed from one such fluid cavity by means of a non-coring needle passed through the skin of the patient and penetrating the septum into one of the respective fluid cavities. This medication is directed through the distal end of the catheter to an entry point into the venous system of the body of the patient.

Blood may also be withdrawn for sampling from the body of a patient through such an access port. This is accomplished by piercing the skin of the patient and one of the respective septums with a non-coring needle and applying negative pressure thereto. This causes blood to be drawn through the catheter into the fluid cavity corresponding to the pierced septum and then out of the body of the patient through the needle.

To prevent clotting thereafter, the withdrawal route is flushed with a saline solution or heparin using again a non-coring needle piercing the skin of the patient and the septum in the same manner as if a medication was being infused.

Both intermittent and continual injections of medication may be dispensed by the access port. Continual access involves the use of a non-coring needle attached to an ambulatory-type pump or a gravity feed IV bag suspended above the patient. The ambulatory-type pump or the IV bag continually feeds the medication or fluid through the needle to the fluid cavity in the access port and from there through the catheter to the entry point into the venous system.

To facilitate locating each respective septum once the access port has been implanted, some access ports incorporate a raised circular ring located about the outer perimeter of the septum. This raised ring enhances the tactile sensation afforded by the subcutaneous septum to the palpating fingertip of a medical practitioner. Alternatively, other access ports have utilized palpation ridges rather than a raised circular ring for substantially the same purpose. The palpation ridges allow the location of the septum to be accurately determined when the access port is subcutaneously implanted.

To preclude reaction with the tissues in the body of the patient, access ports are constructed of non-reactive materials, such as titanium or stainless steel. Although these materials are non-reactive, access ports constructed utilizing titanium or stainless steel materials produce an interfering or blurred image of the body of the patient in the vicinity of the implanted access port when diagnostic imaging techniques such as magnetic resonance imaging (“MRI”), CAT scans, or computerized tomography are used. The blurred region caused by the presence of a metallic access port in the body of a patient extends beyond the access port itself. Therefore, the use of metallic access ports limits the diagnostic imaging techniques that may be used relative to those areas of the body in which an access port is implanted. In place of metallic materials some access ports have been fabricated at least in part from biocompatible plastics.

A further problem relating to the materials for and manufacture of access ports is the deleterious impact of some manufacturing procedures on the fluids which flow through the fluid cavities and related structures located between the fluid cavities and the catheter. During the manufacture of an access port, whether the port is made of metallic or plastic materials, it becomes necessary to form the fluid cavities and exit passageways through which the fluid will be directed into the attached catheter. This manufacturing process often leaves sharp edges, seams and comers in the areas where the fluid cavity is to direct the flow of the fluid through an exit passageway. As blood or other fluids are injected through the septum into the fluid cavity, pressure developed within the fluid cavity tends to cause fluid to flow through the exit passageway. As the fluid in the fluid cavity flows past the sharp edges and comers produced in the manufacture of the access port, turbulence arises, taking the form of a vortex, adjacent to the sharp edges and corners. Some fluids, such as blood, are sensitive to this turbulence, and lysing of the red blood cell component of the injected blood can occur in these turbulent areas.

In addition, the production of the circular fluid cavities often results in the creation of areas within the housing in which fluid flow is retarded. These areas are referred to as dead spaces and usually occur in areas of transition, such as where the bottom of the septum interfaces with the walls of the fluid cavity and where the floor of the fluid cavity meets the exit passageway through which the fluid must flow. As the flow of fluids through dead spaces is retarded, stagnation occurs, resulting in some fluid being trapped within these dead spaces. If the access port is used to withdraw or transfuse blood, blood trapped in these dead spaces may form clots and block the flow of fluid through the fluid cavity.

Moreover, in some prior vascular access ports the internal reservoirs are formed by two plastic parts which are bonded together. This results in an undesirable seam being formed where the adjacent parts abut one another. The inside of the reservoir should be as smooth as possible to help prevent damage to blood cells or the initiation of blood clotting during infusion or withdrawal of blood through the port.

A further problem encountered in the design and construction of access port relates to the positioning of the septums within the housing of the access port. The positioning of the septums within the housing is a compromise between two conflicting objectives. These are the need to separate the septums to such a distance so that the septums may be easily differentiated for the purpose of injection and the need to restrict the overall dimensions of the access port for patient comfort and aesthetics. The distancing of the septums to facilitate their differentiation, however, results in a corresponding distancing of the fluid cavities. This result is at odds with another structural requirement for access ports with plural cavities, namely that the exit passageways from each fluid cavity be closely spaced at the point where the implanted catheter is to be coupled to the access port.

To guide the flow of a fluid from each of the spatially separated fluid cavities into the side-by-side configuration of fluid outflow necessitated by the dimensions of a plural lumen catheter, intermediate structural members have been required. Naturally, this complicates the process of manufacture and increases its cost, as well as the changes of structural failure.

There are several examples of such intermediate members used to resolve the manufacturing constraints imposed upon the construction of a passageway flowing from spatially separate fluid cavities into a side-by-side configuration acceptable by a catheter. One is to produce passageways in the form of bent metal tubes which are then insert molded or welded into the larger body of the access port. The use of such a metal component will interfere with the production of an access port which is free of limits as to the diagnostic imaging techniques that may be used relative to those areas of the body in which an access port is implanted. In addition, the integral nature of such metal outlet passageways raises the possibility of leakage of medication through the interstices between the metal tubes and the body of the access port.

Alternatively, to produce fluid flow from spatially separated fluid cavities into the closely spaced lumens of a catheter, each fluid cavity has been designated with its own spatially separated outlet stem. These outlet stems are then coupled by a hub structure for permanent attachment to the closely spaced lumens of a catheter. This type of arrangement increases the size of the overall access port and its cost of manufacture by adding thereto the necessity of fabricating and assembling of the hub element. Port connections to catheters in this manner are permanent. Accordingly, if the catheter is to be shortened by trimming, that trimming must occur at the distal end of the catheter, and this precludes the use of any type of specially designed tip or valve.

An additional set of problems encountered in the use of access ports relates to the actual connection of the catheter to the access port. This is most commonly effected by securing the catheter to an outlet stem protruding from the housing of the access port. In an attempt to lock the catheter to the outlet stem of the access port, thread-type systems have been developed wherein the catheter is attached to an outlet stem, and the outlet stem is then threaded into the access port. When utilizing this system, however, it is difficult to determine the amount of engagement of the catheter onto the outlet stem. Some catheter connection systems do not allow visual verification of attachment. As a result, leakage and failure can occur.

To overcome this problem, access ports are produced in which the catheter is pre-attached at the factory. While this practice alleviates many of the problems with leakage and failure due to catheter slippage, this system severely limits the type of the catheter usable with the access port. This precludes the use of catheters having specialized distal tips, as the distal end of the catheter is the only end that can then be trimmed to effect its ultimate sizing. For example, catheters utilizing a Groshong.RTM. slit valve at their distal end may not have any of the distal tip of the catheter removed without compromising the catheter.

Thus, there has been a need for an improved vascular access port which overcomes the above-noted problems, and which can be manufactured economically. The present invention fulfills these needs and provides other related advantages.

### SUMMARY OF THE INVENTION

The present invention provides a configurable implantable access port assembly. The access port assembly includes a chamber body defining a fluid chamber, a septum configured to provide ingress and egress to said fluid chamber, and interchangeable body components, wherein said chamber body and said septum are adapted to mate with a plurality of interchangeable body components. Preferably, at least the interchangeable body component may be provided having more than one geometry and or size. Accordingly, a standard sized chamber body and septum may be assembled with body components of differing sizes and/or geometries to provide access ports having different overall sizes and or geometries.

According to a related aspect, the present invention provides an access port having interchangeable components that may be configured and assembled to provide an access port that is tailored in size and/or geometry to a specific application. Accordingly, it is possible to provides the greatest number of different access port configurations with the least, and least expensive, manufacturing tooling.

In yet another related aspect, the present invention provides a kit of individual and interchangeable access port components that may be assembled to provide a completed implantable access port. The kit may include a chamber body defining an open fluid chamber, a body component, and a septum for providing ingress and egress to said fluid chamber. Preferably the kit includes a plurality of body components, the plurality of body components having differing sizes and or geometries.

### BRIEF DESCRIPTION OF THE DRAWINGS

Advantages of the present invention will be apparent from the following description, which description should be considered in conjunction with the accompanying drawings, wherein:

FIG. 1 is a perspective view of an assembled exemplary implantable access port consistent with the present invention;

FIG. 2 is an exploded perspective view of the exemplary implantable access port illustrated in FIG. 1;

FIG. 3 is a perspective view of a second exemplary body portion consistent with present invention;

FIG. 4 is a perspective view of an exemplary implantable access port utilizing the second exemplary body portion consistent illustrated in FIG. 3;

FIG. 5 is a perspective view of a third exemplary body portion consistent with the present invention; and

FIG. 6 is a perspective view of an exemplary implantable access port utilizing the third exemplary body portion illustrated in FIG. 5.

### DESCRIPTION THE INVENTION

The present invention is an access port device including an assembly of interchangeable components. As such, the access port may generally include a fluid chamber body, a body portion, and a septum that may be provided in a non-integral assembly. The various components of the access port may be provided having several different configurations. As such, access ports may be assembled from different body components to achieve implantable access ports having a range of different configurations.

Consistent with one exemplary embodiment of this aspect of the invention, it is noted that, during implantation, it may be desirable to fasten the access port to adjacent tissue to secure the access port against undesired migration from the implanted position. The access port may be provided with suture apertures to facilitate anchoring the access port by suturing the access port to tissue adjacent to the implant site. Securing the port in this manner may allow the access port to be securely stabilized subcutaneously in the intended implantation site. However, different implantation sites in the body may require different configurations of suture apertures, if any at all, and different access port profiles.

Additionally, it is also noted that the size and gender of a patient, as well as the intended implantation site may greatly vary the size, shape, and geometry of the necessary or desired access port. Rather than requiring a completely different access port for each implantation site or application, the access port consistent with the present invention may allow the alteration of only specific components, such as the body portion, in order to accommodate different applications. Therefore, the suture ring may influence the applications for which the access port may be beneficial. However, it should be understood that the suture ring described below may be simply an exterior body portion lending itself to the overall profile of the access port, and may exist independent of any suturing facility of the access port.

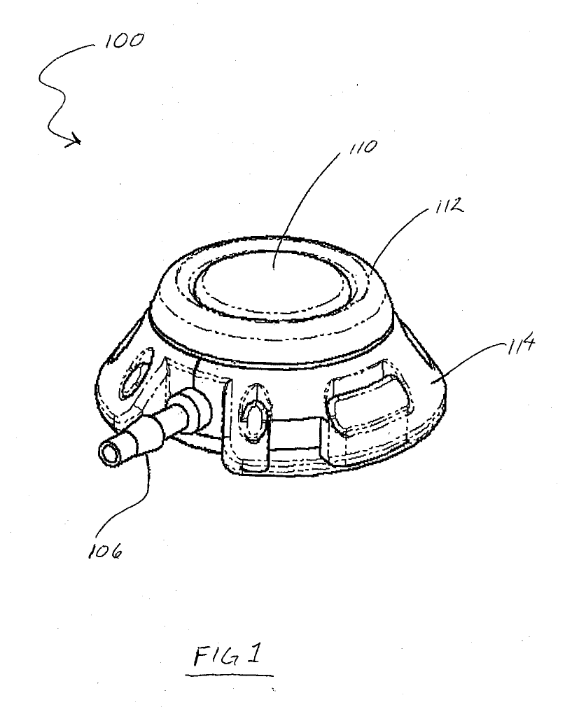

Referring to FIG. 1, a first exemplary embodiment of a single access port device 100 consistent with the interchangeable assembly is illustrated. According to the exemplary embodiment, the access port 100 includes a septum 110 surrounded by a cap ring 112 that forms an upper rim of the access port 100. The cap ring 112 may be disposed on a top surface of an interchangeable body component, which in the case of this embodiment is a suture ring 114. The body component 114 may generally defines the overall profile and geometry of the access port 100. A stem 106 may be provided extending from a sidewall of the access port device 100 and in fluid communication with a fluid chamber internal of the access port 100.

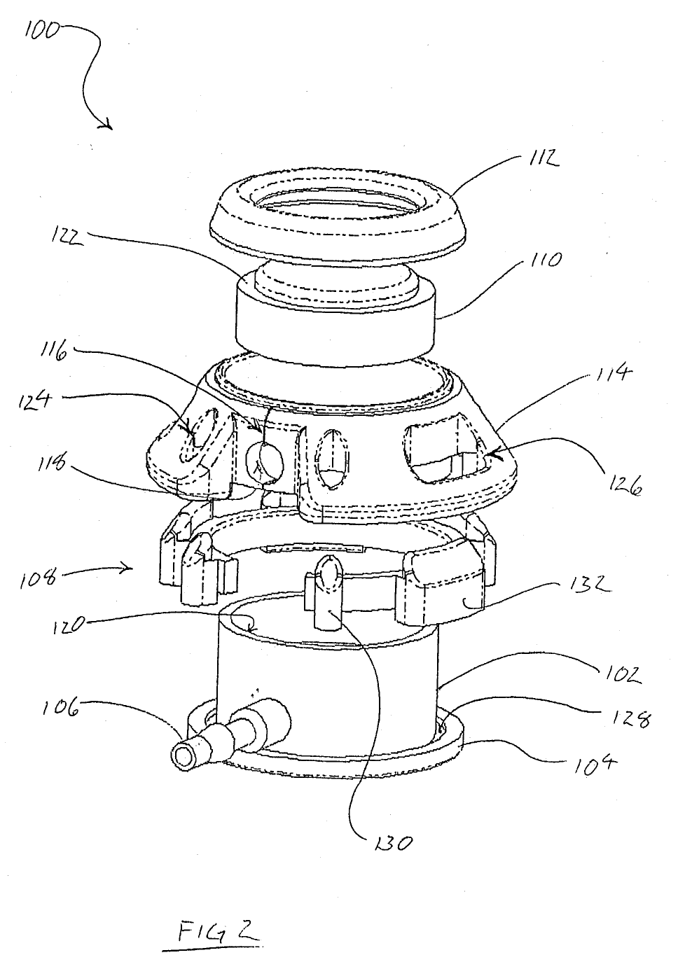

Turning to FIG. 2, the exemplary access port 100 is illustrated in an exploded view. The access port assembly 100 generally includes a fluid chamber body 102, that defines an open-ended fluid chamber; the suture ring 114; and a septum 110 that may permit self sealing ingress and egress of a needle. As illustrated, the access port 100 may also include a suture plug 108 disposed at least partially between the chamber body 102 and the suture ring 114. Also, the access port 100 may include a cap ring 112 that is disposed at an upper portion of the access port 100, and preferably surrounds at least a portion of the septum 110.

Referring to the illustrated embodiment, the chamber body 102 may be a generally tubular member having a bottom portion 104, and a stem 106 extending from a sidewall of the chamber body 102, in fluid communication with an interior thereof. The suture plug 108 may be disposed around the chamber body 102 such that the opening in the suture plug 108 accommodates the stem 106. In the illustrated embodiment, the suture plug 108 may include a plurality of plug members 130 and 132. As illustrated the plug members may be cylindrical members 130 or rectangular members 132. According to the exemplary embodiment, the upper portions of the plug members 130, 132 may be contoured corresponding to a contour of the suture ring 114. The plug members 130, 132 at least partially fill the openings in the suture ring 124, 126 in order to prevent migration of tissue or biometric material into the suture apertures 124, 126. The suture ring 114 may in turn be positioned over and around both the chamber body 102 and the suture plug 108. As illustrated, the suture ring 114 may include an aperture 118 permitting the stem 106 to extend there through. Desirably, a marginal region of a bottom surface of the septum 110 may be at least partially received in the sidewall 120 of the chamber body 102. Advantageously, a bottom surface of the septum 110 may contact an inwardly extending feature of the chamber body (not shown). Finally, the cap ring 112 may be disposed on the septum 110, wherein a bottom surface of the cap ring 112 may be at least partially coextensive with an upper marginal region 122 of the septum 110, compressing the septum 110 against the sidewall 120 of the chamber body 102.

The chamber body 102 may be formed of any biocompatible material and configured having a bottom portion and a wall portion defining a fluid chamber having an access for the ingress and egress of a needle. According to the illustrated exemplary embodiment, the chamber body 102 may be configured to have a generally round, tubular geometry and include a bottom portion 104. In one example, the chamber body may be formed as a cup. Additionally, the chamber body 102 preferably includes an exit port in fluid communication with the stem 106. Advantageously, the chamber body 102 is formed of a metallic material, such as stainless steel, titanium, or other biocompatible metal. Alternatively, the chamber body 102 may be a molded biocompatible plastic material. Furthermore, the chamber body 102 may include a metallic or ceramic cup disposed at the bottom of the fluid chamber to resist scratching and/or debris release as a result of needle impact. An exemplary configuration of this aspect is embodied in commonly assigned U.S. patent application Ser. No. 09/582,406, filed on Jun. 23, 2000, the teachings of which are incorporated herein by reference.

The suture plug 108 is an optional component that may be employed to prevent the ingrowth and/or accumulation of tissue or other biometric material in the suture apertures 124, 126 of the suture ring 114. In keeping with this objective, preferably the plug members 130, 132 are disposed in the suture apertures 124, 126. The suture plug 108 may be formed of an elastomeric material, e.g., silicone, such that the plug members 130, 132 may be penetrated with a suture needle and receive a suture passing through the plug members 130, 132. The elastomeric material may conform around the a suture passing therethrough

Desirably, the suture plug 108 may be sized to fit over the outer circumference of the chamber body 102, and may be at least partially retained by frictional engagement between the suture plug 108 and the chamber body 102. The suture plug 108 may optionally be more securely coupled to the chamber body 102, for example by being disposed in a shallow groove in the outer wall of the chamber body 102, or in a groove 128 in the base portion 104. Alternatively, the chamber body 102 and the suture plug 108 may include interacting protrusions and recesses for securing the two components. Still alternately, the suture plug 108 and the chamber body 102 may be coupled by welding or adhesive bonding using biocompatible adhesives, using techniques known in the art and/or described herein.

The body component-suture ring 114 is preferably a molded plastic component, advantageously an injection molded biocompatible plastic article. As illustrated, the suture ring 114 may include a split, indicated at 116 in FIG. 2, bisecting the aperture 118. This spit 116 may not only facilitate molding of the suture ring 114, but may also allow the suture ring 114 to be positioned over the suture plug 108 and the chamber body 102 by separating forcing the split 116 open to an extent whereby the stem 106 may be received via the split 116 to extend through the aperture 118. The suture ring 114 may also include suture apertures 124 and 126 corresponding to plug members 130, 132 disposed on the suture plug 108, as mentioned above. The apertures 124 and 126 may include wall portions extending downwardly from an upper surface of the suture ring 114, wherein the wall portions may be configured to at least partially receive the plug members 130, 132 or the suture plug 108.

The suture ring 114 may be assembled to the chamber body 102 and/or the suture plug 108 by interacting and/or mating mechanical securement means, such as snap-fit, tongue and groove features, bead and channel features, press fits, etc. respectively on the suture ring 114 and at least one of the suture plug 108 and the chamber body 102. One example may include an undercut, or similar feature on the suture ring 114 may that can engage with a peripheral edge of the base portion 104 of the chamber body 102. Numerous other possible ways of assembling the suture ring 114 to the chamber body 102 and/or the suture plug 108 will be readily apparent to those having skill in the art, including methods such as welding, biocompatible adhesives, and combinations of these.

In addition to, or as part of assembling the suture ring 114 to the chamber body 102 and/or the suture plug 108, the split 116 in the suture ring bisecting the aperture 118 may be joined. Joining the suture ring 114 across the split 116 may be accomplished using mechanical features, such as snap-fits or press fits, welding, biocompatible adhesive, etc. Joining the split 116 may increase the security of the assembly, for example by securing the suture ring 114 to the stem 106, or by preventing circumferential snap-fit on the suture ring from opening up as from the separation of the split 116.

The septum 110 may be formed of an elastomer, such as silicone, that is semi-permeable, in that the septum 110 may allow the ingress and egress of needles to deliver fluid to the fluid chamber. Consistent with previous disclosure, the septum 110 may be provided with tactile features allowing percutaneous identification of a specific port.

The cap ring 112 is desirably formed from a metallic or ceramic material to reduce possible scratching and/or debris being introduced through the septum 110. However, the cap ring 112 may also be formed from more economical materials, such as biocompatible polymers. The cap ring 112 may include a rounded or angled upper surface to urge a needle downward toward the septum 110, thus preventing errant entry of needles within the septum. Advantageously, cap ring may be coupled to the suture ring 114 using mechanical features, such as tongue and groove features, and/or biocompatible adhesive or welding.

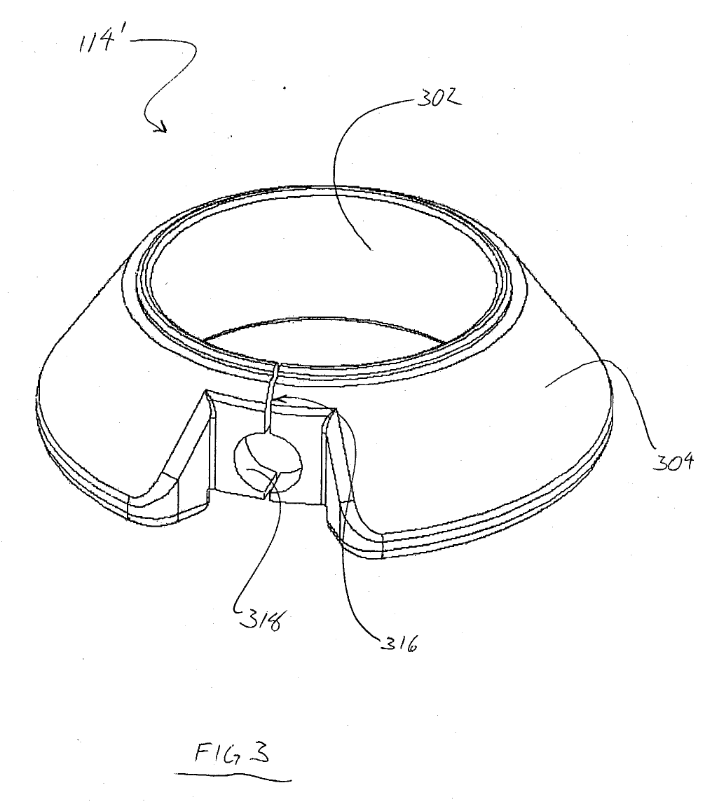

FIG. 3 depicts a second exemplary embodiment of a body component 114′ consistent with the present invention. The body component 114′ includes an inner wall 302 configured to receive a chamber body 102 therein. In the illustrated embodiment includes a sloping upper surface 304 that does not include any suture apertures therein. Accordingly, an access port using this exemplary body component 114′ may be subcutaneously implanted in an unrestrained manner. As with the previous embodiment, the body component 114′ may include an aperture 318 and a split 316 for accommodating a stem.

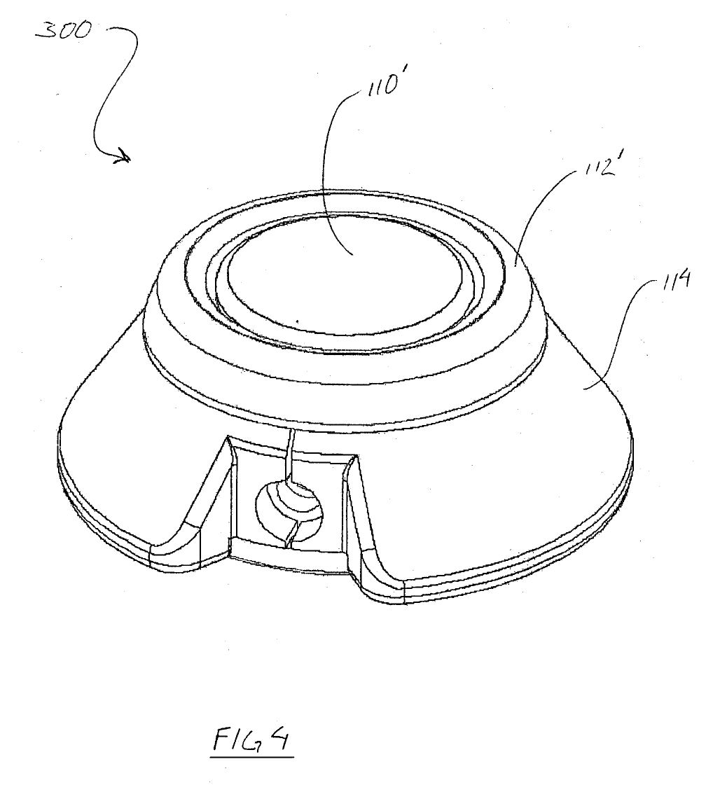

An exemplary assembled implantable access port 300 is illustrated in FIG. 4 utilizing the second exemplary body component 114′. The access port 300 may generally include the body component 114′ having a septum 110′ and a cap ring 112′ assembled thereto, as well as a chamber body (not shown). The several components may be assembled using mechanical features, welding, adhesive bonding, and combinations thereof. Following an objective of the invention to provide an access port 300 that is an assembly of interchangeable components, the septum 110′, cap ring 112′, and chamber body may be of the exact, or similar variety as described in the context of the first exemplary embodiment. It should, however, be appreciated that a suture plug is not required in the instant embodiment.

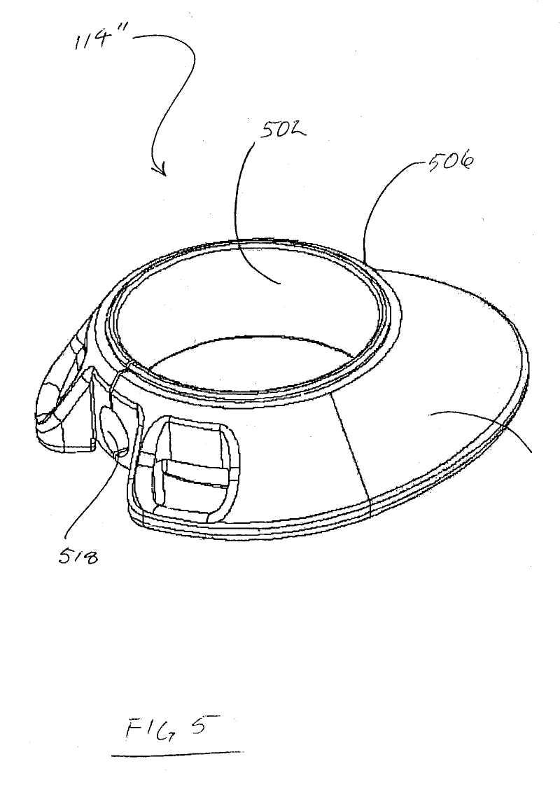

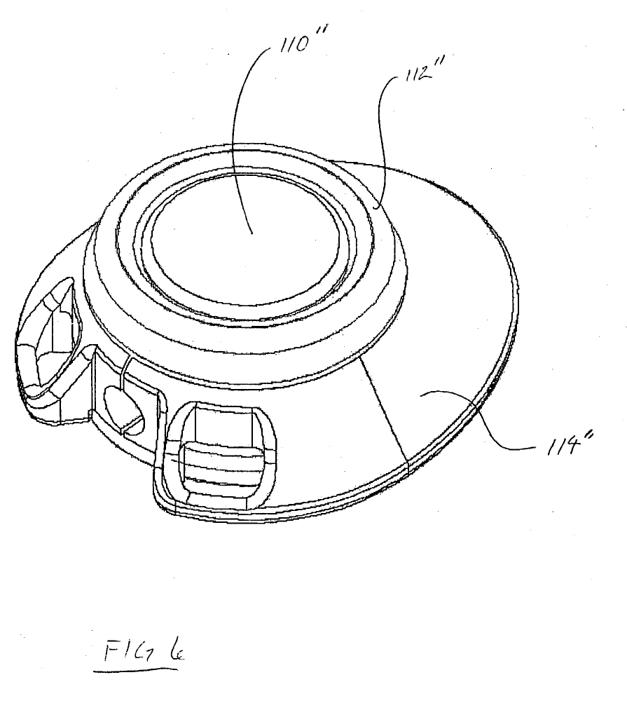

FIGS. 5 and 6 illustrate yet another exemplary alternative embodiment of the invention. A third exemplary embodiment of the body component 114″ is provided having an inner wall 502 defining an opening for receiving a chamber body. An upper rim 506 of the body component 114″ may be provided with, for example, a tongue feature for attaching a cap ring 112″ and/or septum 110″ thereto, as shown in FIG. 6. The upper surface 504 of the body component 114″ tapers downwardly and outwardly from the rim 506 in an eccentric manner, providing an oblong configuration that the body component is elongated away from the aperture 518. It should be appreciated that the body component 114″ may be adapted to various alternative eccentric and or non-round configurations. Additionally, while the exemplary embodiment illustrated in FIGS. 5 and 6 is not shown to include suture apertures, those having skill in the art should readily appreciate that suture apertures and various similar features maybe be incorporated.

While the several illustrated exemplary embodiments herein have depicted various configurations of the body component, it should be appreciated that the numerous other components, chamber body, septum, cap ring, etc., may similarly be provided having an array of different configurations, thereby increasing the number of available configurations of the access port assembly. Advantageously each of the embodiments of the various components may adhere to some standard critical dimensions such that various embodiments of a component are interchangeable in the access port assembly with the other components. For example, the various body components may be configured to receive a standard sized chamber body, and the various septums may be configured to be employed with a standard sized body component, cap ring, and chamber body. However, various embodiments of the several components may be provide to only function with a subset of embodiments of the other components, yielding less than complete interchangeability.

Consistent with an optional embodiment, individual components of the access port may be selected and assembled by a doctor or technician to tailor the access port to a specific application. Advantageously, the access port may be assembled on a demand basis from stock components, rather than relying on preassembled components configured for a general variety of applications.

The invention herein facilitates relatively easy, cost effective manufacture, as well as improved versatility. Each of the individual components may be susceptible to cost effective mass-production, for example by generally conventional injection molding, or insert injection molding, metal injection molding, casting, etc. Additionally, body components having a wide variety of different profiles may be provided that can accommodate or be assembled to a “standard” chamber body. Therefore, it may be possible to provide access ports having a numerous configurations, facilitating placement at different locations of the body, by only changing one component of the assembly. In additional, not illustrated embodiments, the stem may be provided as a T or Y stem, whereby two chamber bodies may be fluidly coupled to a single stem. Consistent with this aspect, a suture ring may be provided configured to receive two chamber bodies, thereby providing multiple port access device incorporating the interchangeable aspect of this aspect of the invention without necessitating the production of two-port chamber bodies.

Thus, it is apparent that there has been provided an implantable vascular access device that satisfies the objectives set forth herein. Those skilled in the art will recognize that the present invention is subject to modification and/or alterations, all of which are deemed within the scope of the present invention, as defined in the appending claims.

1. An implantable access port assembly comprising:
a chamber body including a base, an upstanding wall extending from said base defining a fluid chamber, and an exit port in said chamber body in fluid communication with said fluid chamber;
an interchangeable body component comprising an opening configured to receive said chamber body therein; and
a septum disposed configured to define a top of said fluid chamber;
where is said chamber body and said septum are adapted to mate with a plurality of said body components.

2. An implantable access port according to claim 1 further comprising an annular cap ring securable to said body component and sized to at least partially cover a marginal edge of said septum.

3. An implantable access port according to claim 1 wherein said body component is a suture ring having at least one suture aperture extending therethrough.

4. An implantable access port according to claim 3 further comprising a suture plug including at least one plug member configured to be received in said at least one suture aperture.

5. An implantable access port according to claim 1 wherein said chamber body comprises a biocompatible metal.

6. An implantable access port according to claim 1, wherein said chamber body comprises a biocompatible plastic.

7. An implantable access port according to claim 6, said chamber body further comprising a metallic insert member covering at least a portion of a bottom of said fluid chamber.

8. An implantable access port according to claim 1 wherein said body component comprises a molded plastic component.

9. An implantable access port kit comprising, as individual components:
a chamber body including a wall defining an open ended fluid chamber;
an interchangeable body component including a recess configured to receive said chamber body;
a septum sized to close said open ended fluid chamber defined by said chamber body, wherein said chamber body and said septum are adapted to mate with a plurality of said interchangeable body components.

10. An implantable access port kit according to claim 9 comprising a plurality of interchangeable body components, said plurality of interchangeable body components having different geometries, whereby mating said chamber body and said septum with different body components yields an access port having a different geometry.

11. An implantable access port kit according to claim 9 comprising a plurality of interchangeable body components, said plurality of interchangeable body components having different sizes, whereby mating said chamber body and said septum with different body components yields an access port having a different size.

12. An implantable access port kit according to claim 9, wherein said interchangeable body component has a generally circular profile and tapers from a relatively thick portion adjacent to said recess to a relatively thin marginal edge.

13. An implantable access port kit according to claim 9, wherein said interchangeable body component has a generally oval profile and tapers from a relatively thick region adjacent said recess to a relatively thin marginal edge.

14. An implantable access port kit according to claim 13, wherein said relatively thick region is eccentrically located in said interchangeable body component.

15. An implantable access port kit comprising, as individual components:
a chamber body including a wall defining an open ended fluid chamber;
an interchangeable suture ring including a recess configured to receive said chamber body and at least one suture aperture extending through said suture ring;
a septum sized to close said open ended fluid chamber defined by said chamber body, wherein said chamber body and said septum are adapted to mate with a plurality of said interchangeable suture rings.

16. An implantable access port kit according to claim 15 comprising a plurality of interchangeable suture rings, said plurality of interchangeable suture rings having different geometries, whereby mating said chamber body and said septum with different suture rings yield an access port having a different geometry.

17. An implantable access port kit according to claim 15 comprising a plurality of interchangeable suture rings, said plurality of interchangeable suture rings having different sizes, whereby mating said chamber body and said septum with different suture rings yield an access port having a different size.

18. An implantable access port kit according to claim 15, wherein said interchangeable body component has a generally circular profile and tapers from a relatively thick portion adjacent to said recess to a relatively thin marginal edge.

19. An implantable access port kit according to claim 15, wherein said interchangeable body component has a generally oval profile and tapers from a relatively thick region adjacent said recess to a relatively thin marginal edge.

20. An implantable access port kit according to claim 19, wherein said relatively thick region is eccentrically located in said interchangeable body component.
